(19) United States
(12) Patent Application Publication
(10) Pub. No.: US 2004/0034397 A1
(43) Pub. Date: Feb. 19, 2004

(54) Method and apparatus for treating skin disorders using a short pulsed incoherent light

(76) Inventor: J. T. Lin, Oviedo, FL (US)

Correspondence Address: J. T. LIN, 4532 Old Carriage Trail, Oviedo, FL, 32765, US

(21) Appl. No.: 10/217,447

(22) Filed: Aug. 14, 2002

(51) Int. Cl. A61N 1/00

(52) U.S. Cl. 607/94

(57) ABSTRACT

A method and apparatus for treating various skin disorders of psoriasis, vitiligo, eczema, dermatitis and acne is presented. An apparatus for the treatment of skin disorders includes a power supply, a light source, a reflecting mirror, a filter, a fiber delivery unit and a hand piece. A spectra selector using a filter or reflecting mirror is used to select a UV spectrum about (270-320) nm or a blue spectra about (405-435) nm which is delivered to the treated skin area by a light guide and a hand piece. A power supply is used to generate pulsed light of about (0.01-500) microseconds and a repetition rate of (1-500) Hz. Adjustable light spot size of 0.5×0.5 cm to 10×10 cm is proposed for efficient treatments of both small and large areas such that only the disordered areas are selectively treated whereas the exposure of the healthy areas is minimized. Short pulsed incoherent with high peak power is more efficient than conventional continuous wave UVB or blue light and shorten the treatment time by a factor of (5-20) times. A singe device providing multiple spectra for various applications is proposed by a simple switching of hand piece or filter.

### FIELD OF THE INVENTION

The present invention relates to a method and apparatus for treating skin disorders including psoriasis, vitiligo, eczema, dermatitis or acne. More particularly, the invention relates to treating the skin disorders by using a pulsed incoherent light with ultraviolet or blue wavelength.

### BACKGROUND OF THE INVENTION

Skin disorders including psoriasis, vitiligo, eczema, dermatitis and acne are relatively common skin disease that affect about 3% of the population. Prior art treatments of these skin diseases include the use of a topical drug and the application of light to the disorder areas. The topical drug treatments produce acceptable results but are limited in their efficiency and the length of time of relief for the patient.

Phototherapy treatments using continuous wave (CW) ultraviolet incoherent light in the UVA (320-400) nm and UVB (270-320) nm ranges has been used extensively. This method of treatment offers a very limited relief to the patient and is used on a very frequent basis (typically 3-4 times a week). Moreover, it provides limited clinical improvement and there is a risk of skin cancer due to the exploring of both healthy and disordered areas. It is known in the prior art to use incoherent light in medical application for therapeutic uses such as treatment of skin disorders. For example, U.S. Pat. No. 4,298,005 to Mutzhas describes a CW ultraviolet lamp with cosmetic, photobiological, and photochemical applications which has a very low power delivered to the skin, about 0.15 W/cm.sup.2, which may not be efficient and may require many treatments (over 35 treatments) for psoriasis. Conventional CW UVB treatment may take longer than 60 minutes for large area of psoriasis in legs and hands because of its low peak power which requires few minutes in each spot of treatment.

Another skin disorder called acne lesion which is currently treated by a CW blue light which takes 10-minute in each explored area on face or back. Topical acne medications also have been used but usually irritating to the skin.

Another prior art in U.S. Pat. No. 6,413,268 by Hartman proposed the use of targeted UVB incoherent light for the treatment of psoriasis, which again is a CW radiation generated from an arc lamp and still has many drawbacks: low peak-power (maximum of 4.8 W), long warm-up time and limited to three major wavelengths at 302, 312, 318 and 365 nm and thus its applications are limited to psoriasis-type diseases. The procedure may may still take long time, due to its low peak power (about 1-5 W) and its limited light spot size (about 2×2 cm). The energy per pulse required in the CW light is much higher (about 200-500 mJ) than that of a pulsed light proposed in the present invention (about 0.5-20 mJ). Prior art of Eckhouse (U.S. Pat. No. 5,405,368) proposed a pulsed incoherent light, in non-UV range of (400-1100) nm which however has no reported clinical significance.

A prior art in U.S. Pat. No. 5,955,840 used a pulsed XeCl excimer laser at 308 nm to treat psoriasis. Another prior art for treating psoriasis (U.S. Pat. No. 5,312,395) used a pulsed dye laser operating at a wavelength of 585 nm, with a pulse duration of 0.4 micron seconds and fluency in the range of 6 to 10 J/cm.sup.2 (system made by Candela, USA). While laser treatments were generally effective, one significant drawback is the small beam spot size (5-10 mm) which makes the treatment very inefficient since psoriasis typically appears on large areas of the skin. A typical area of whole leg may take longer than 60 minutes for each visit. Furthermore, a laser at a specific wavelength (308 nm or 585 nm) to treat a certain type of skin pigmentation, disorder may not be absorbed efficiently by skin having a different type of disorder. In addition, lasers are usually complicated, expensive to manufacture, limited by amount of power delivered and difficult to maintain.

Accordingly, a method and apparatus for treating multiple skin disorders including psoriasis, vitiligo, eczema, dermatitis (require UVB wavelength) and acne (requires a blue wavelength) is desirable. Additionally, adjustable spot size from 0.5×0.5 cm to 10×10 cm will preferably be required for treating both small area of vitiligo and relatively large area of psoriasis and others. A short pulse of (0.01-500) microseconds, incoherent light in the UVB range of (270-320) nm and blue range of (405-435) nm are preferably required to overcome the drawbacks of the prior art treatments. Finally, a single device providing multiple applications by selection of either UV or blue spectrum is proposed in this invention. The proposed short pulse having a peak power in KW range will shorten the treatment time for each spot to few seconds which is (20-100) times faster than all prior arts using a CW incoherent light. The present invention proposed “localized” or “targeted” spot treatment plus the use of KW peak-power light provides us a unique method for safe and efficient phtotherapy which significantly reduce the number of treatments required and exposure time in each treated spot without being limited to the damage threshold of the adjacent health tissue.

### SUMMARY OF THE PRESENT INVENTION

One preferred embodiment of the invention for treating skin disorders of psoriasis, vitiligo, eczema and dermatitis includes generating one or more pulses of incoherent electromagnetic energy (light) and directing this light to a region of tissue to be treated. The pulsed light may have a pulse duration of about (0.01-500) microseconds, energy per pulse on the target of (0.2-20) m and peak-power of (0.1-5) KW depending on types of skin and diseases. Another preferred embodiment is to use means of spectrum selection including a filter absorbing unwanted spectra and selecting wanted UV spectra and a coated 45-degree reflection mirror having high reflection of the UV spectra. The energy/pulse and the number of pulses are selected to control a treatment parameter and optimize the treatment. In one embodiment a large spot size 2×2 cm to 10×10 cm is created to treat large area of psoriasis and a small spot of about 0.5×0.5 cm to 1×1 cm for the treatment of vitiligo or eczema such that only the disordered areas are selectively treated, whereas the exposure of the healthy areas is minimized. Because of the short pulse duration with very high peak power (in the order of KW range), the treating time of each spot will be in the order of few seconds which is about (5-50) times faster than that of the CW low-power (1-5 W) UVB light as proposed in the prior arts. The present patent also proposes the use of pulsed blue-light with spectrum of (405-435) nm to treat another skin disorder called acne lesion which is currently treated by a CW light taking about 10-minute in each spot of treatment. This treatment may be shortened to about (5-10) seconds when a pulsed light is used.

The present invention proposed “localized” or “targeted” spot treatment plus the use of KW peak-power light provide us a unique method for safe and efficient phtotherapy which significantly reduce the number of treatments required and exposure time in each treated spot without being limited to the damage threshold of the adjacent health tissue. We further note that the abnormal tissue typically has a much higher withstand radiation power than that of normal tissue, using a pulsed light with high peak-power therefore is highly desired for maximum therapeutic effects. Unlike a laser light (at 308, 585 nm) or a CW incoherent used in the prior arts (which produced a discrete major radiation wavelengths at 302, 312 and 318 nm), the proposed pulsed light in this invention produces continuous spectrum covering the range of (200-1100) nm in which one can select either UVB band (270-320) nm or blue band (405-350) nm in a single lamp and power supply to achieve maximum therapeutic effects with minimum side effects (such as erythermal or carcinogentic). The energy per pulse required in the CW light is much higher (about 100-500 mJ) than that of a pulsed light proposed in the present invention (about 0.2-20 mJ), therefore the CW light is less efficient and causing more thermal damage to the adjacent health tissue.

An apparatus for the treatment of skin disorders includes a power supply, a light source, a reflecting mirror, a filter, a fiber delivery unit and a hand piece. A pulsed light source that produces incoherent radiation in from UV to near infrared (IR) range of wavelength, (200-1100) nm, is placed in the housing. A curved reflector reflects and directs the light to the filter and fiber delivery unit. A filter removes or attenuate unwanted portions of the spectrum in the visible and IR range. And only keep the narrow band UVB spectrum of (270-320) nm or blue-spectrum of (405-435) nm. This UVB or blue spectrum may be further selected by using at least one 45-degree high-reflecting mirror which reflects the wanted UVB (270-320) nm or blue (405-435) nm and let the unwanted portion of the spectra pass through. In another embodiment a light guide is placed to deliver the UVB or blue light to the hand piece and then to the area to be treated. The light guide may be a UVB (or blue) transparent optical fiber or a flexible light guide filled with UVB (or blue) transparent liquid or an articular arm. A power supply that includes a pulse generator is provided to power the light source and controlled by a microprocessor for the power output and repetition rate (or number of pulses per second). Another preferred embodiment is to integrate the lamp (or the light source) in the hand piece such that the delivery light guide will not be needed.

Other principal features and advantages of the invention will become apparent to those skilled in the art by the following drawings, the detailed description and the claims. It is to be understood that the invention is not limited in its application to the details of construction and the arrangement of the components set forth in the following description or illustrated in the drawings. The invention is capable of other embodiments or being practiced or carried out in various ways. Also, it is to be understood that terminology employed herein is for the purpose of description and should not be regarded as limiting.

### BRIEF DESCRIPTION OF THE DRAWINGS

FIG. 1 is a schematic illustration of one preferred embodiment consisting of a power supply, a housing and a fiber deliver unit to guide the light to the hand piece.

FIG. 2 is a schematic of the hand piece consisting of a filter or a reflecting mirror and adjustable aperture to control the output light spot size on the treated area.

FIG. 3 is a schematic illustration of another preferred embodiment having the light source integrated in the hand piece and no fiber unit is required.

### DETAILED DESCRIPTION OF THE PREFERRED EMBODIMENTS

The invention relates to a method and apparatus for treating various skin diseases including psoriasis vitiligo, atopic eczema, seborrheic dermatitis and acne. Generally, the invention uses pulsed light, preferably incoherent, in the ultraviolet range, that causes the clearance of psoriatic plaque and other skin abnormal cells. In accordance with one preferred embodiment, the apparatus includes a light source that provides electromagnetic radiation that penetrates into the skin, and reaches depths of the order of 0.5 mm or more. Preferably, the fluency of the light will be sufficient to enable to kill the abnormal cells. The light is applied to selected skin disorder area by using adjustable light spot size of 0.5×0.5 mm to 10×10 cm for small area and large area treatments respectively. Large spot size of the light will preferably be large enough to enable efficient treatment of large areas of skin.

An apparatus in accordance with one preferred embodiment of the invention includes a high energy, pulsed, incoherent light source, such as a flash lamp. A suitable flash lamp will generate pulsed light from UV to near infrared range of wavelength, or (200-1100) nm range. The apparatus includes a fixed filter system which absorbs the radiation spectrum, thus reducing the amount of electromagnetic radiation of damaging wavelengths that reaches the skin. The selection of the variable filter (and thus the wavelength of the transmitted light) will allow the optimal treatment to be provided. Spectra selection may also be achieved by one or a pair of coated mirror which is highly reflecting the wanted spectra and highly transparent to the unwanted spectra at an incident angle of 45 degrees. UVB spectra of (270-320) nm or blue spectra of (405-435) nm will be selected depending on the type of skin diseases will be treated. UVB will be good for psoriasis and vitiligo type diseases and blue light will be good for acne lesion. A simple switch of hand piece or exchanging of the filter/mirror will produce either UVB or blue light for different applications.

The light is directed to the skin through an opening in a housing that contains the flash lamp and the filter system in one embodiment. In another embodiment a light guide connected to the housing and in contact with, or in the vicinity of, the skin is used to direct the light to the treatment area.

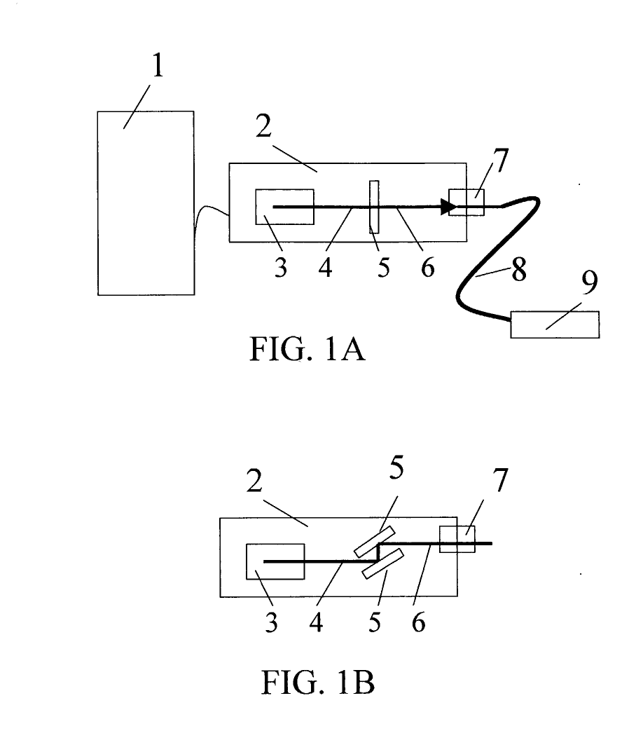

Referring to FIG. 1, a treatment device is schematically shown and includes a power supply 1, a housing 2, a fiber coupler 7 and a hand piece 9. The housing 2 consists of a light source 3 producing incoherent light with wavelength 4 which is converted to UVB (or blue) spectrum 6 by a filter 5. The UV light at (270-320) nm, or blue-light at (405-435) nm, is coupled to the fiber 8 by a coupler 7 and the fiber 8 is further connected to a hand piece 9. The power supply 1 includes a pulse generator is provided to power the light source and controlled by a microprocessor for the power output and repetition rate (or number of pulses per second). The preferred embodiment, light source 1 consists of a flash lamp filled by preferably xenon gas or mixed with other gas such as mercury, and a discharging cathode and anode and probes. Light source 3 may be an incoherent light source such as a xenon gas filled flash lamp which are recently developed by Hamamatsu, Japan and Xenon Corp. USA. The intensity of the flash lamp output is proportional to the input energy. The preferred embodiment for the input energy is about (0.2-5.0) J with an output efficiency of about (10%-30%). The preferred embodiment of the invention includes pulse duration of (0.05-500) microseconds and repetition rate of (1-500) Hz. The UV light after the filter include an energy per pulse of about (0.2-20) mJ and peak-power of (0.1-5.0) KW depending on types of skin and diseases such that enough peak power density (or fluency) is produced for large area treatment. For example, at energy per pulse of 5 mJ operated at 100 Hz and pulse duration of 4 microseconds will provide an average-power of 0.5 W (for one second treatment), peak power of 1.2 KW and fluency of 5 mJ/cm.sup.2 (for a spot size of 10×10 mm). We note that the pulsed light peak power of 1.2 KW is about (100-1000) times higher than that of a typical CW UVB-light proposed in prior arts. This is one of the key features of the present invention to use a short pulsed light for efficient and fast treatment. Therefore the preferred typical treatment time for each treated spot of the present invention is about (0.5-5) seconds versus (20-60) seconds in conventional CW UVB-light treatment of psoriasis and about 10-minutes for acne treatment using blue CW light. The total treatment time or the numbers of visit for a compete curing therefore may be shortened by a factor of (5-20) times when a pulsed light proposed in this invention is used.

Referring to FIG. 1B, an alternative for the selecting of UV spectrum is to use at least one reflecting mirror 5 having a 45-degree highly reflecting the wanted UV B (or blue) spectrum and highly transparent to the unwanted visible or infrared spectra.

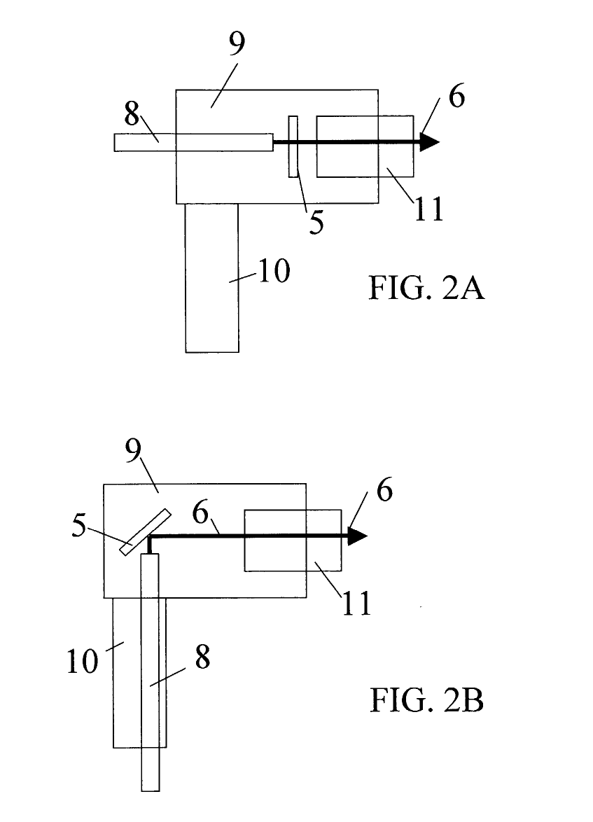

FIG. 2 shows the preferred embodiment of the invention includes a hand piece 9 consists of a fiber 8, a filter or reflector 5, a handle 10 and a light spot controller 11 which is contacting to the surface of the treated area. The light controller 11 is preferably a tube having a square or a rectangular shape with an aperture area of 0.5×0.5 cm to 10×10 cm to be used for small and large area, respectively. This spot controller 11 is inserted to the end of hand piece 9 and it is a exchangeable for various size and for sterilization after the treatments. The shape of the spot controller and its size may be varied. This is another key features of the present invention to use a large spot (up to 10×10 cm) UVB (or blue) incoherent light for efficient and fast treatment which is not available by a laser proposed in the prior arts which have a limited laser spot size to about 0.5×0.5 cm. The spot size controller is attached to the housing and can be easily exchanged for various size. The apertures of the controller may also be electrically controlled by a shutter with pinholes at various size.

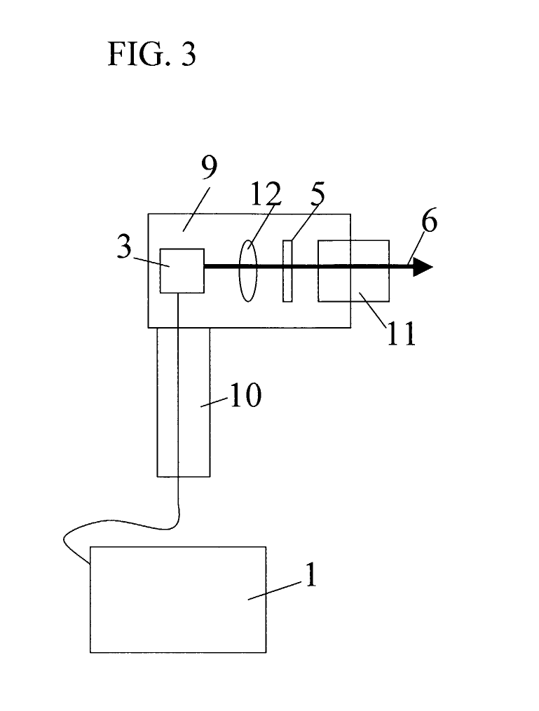

FIG. 3 shows an alternative embodiment for the hand piece where the light source 3 is integrated into the hand piece such that no light guide or fiber is needed. In FIG. 3, the housing 9 has further include at least one focusing optics which collects or re-collimates the light to the spot controller 11 contacting to the surface of the treated skin. The similar reflecting mirror 5 shown in FIG. 2B may also be used in FIG. 3 to replace the filter 5. Another alternative embodiment for the hand piece is to further use a quartz light guide with a spot size of few mm to few cm, which is attached to the end of the spot controller and directly contact to the treated surface. The quartz shall be highly transparent to the UV spectra (270-320) mm or blue spectra (405-435) nm selected. With hand piece integrated by different filter (or reflecting mirror), a switching of the hand piece shall provide us either a UV or a blue spectrum for various applications. Switching of the spectra selector may also be done electrically or by a motorized device which holding the mirror or filter. Because the power supply and light source produce both UV and visible spectrum, a single device with two set of hand piece will be able to perform multiple applications by a simple one step switch either manually or electrically. The number of pulses applied to the treated area may be controlled by a footswitch of other means of turning on-off the lamp power, because there is no warming time needed in a flash lamp device. The exposure time can also be preset by a microprocessor.

The treatment is accomplished by coagulation of hemoglobin and overheating of tissue with abnormal pigmentation. However, to avoid overheating of normal epidermis and decrease pain, a UV absorbing gel may be applied on treated skin surface. Application allows the epidermis to cool during a pulse and to avoid adverse effects if the light pulse duration is approximately the same or shorter as the cooling time of the epidermis. In order to increase the cooling effect the gel may be previously cooled down. Alternatively, the gel may be cooled after it is applied to the skin. The proposed short pulse of (0.01-500) microseconds shall also minimize the risk of overheating to both abnormal and normal skins, particularly when the most preferred embodiment of about (3-5) microseconds pulse is used.

The adjustable light spot size of 0.5×0.5 cm to 10×10 cm also provide a tool for treating the abnormal areas with various shapes and dimensions and minimize the damage to the adjacent health tissue, even a high peak-power (KW range) is used. Without this light spot size selection, the efficiency and peak-power used in the treatment may be limited to what the adjacent health tissue can withstand. Therefore the proposed “localized” or “targeted” spot treatment plus the use of KW peak-power light provide us a unique method for safe and efficient phtotherapy which significantly reduce the number of treatments required and exposure time in each treated spot. Furthermore the selected UVB band width range of (270-320) nm and blue-light of (405-435) nm provide a wider coverage of various skin type and diseases than that of a laser which typically has a narrow single wavelength of either 308 nm (using a XeCl excimer laser) or 585 nm (using a dye laser). Neither of these lasers can treat acne. In the present invention, a single device may treatment multiple skin diseases by a simple switching of the hand piece or exchanging of the filter/mirror inside the hand piece.

It should be apparent that there has been provided in accordance with the present invention a method and apparatus for treating skin disorders that fully satisfy the objectives and advantages set forth above. Although the invention has been described in conjunction with specific embodiments thereof, it is evident that many alternatives, modifications and variations will be apparent to those skilled in the art. Accordingly, it is intended to embrace all such alternatives, modification and variations that fall within the spirit and broad scope of the appended claims.

I claim:

1. A method of treating skin disorders comprising the steps of:
generating at least one pulse of incoherent electromagnetic energy having a pulse duration of about (0.01-500) microseconds,
selecting a wanted spectrum being predetermined,
and directing said pulse of incoherent electromagnetic energy to the area having said skin disorders;
whereby said skin disorders including psoriasis, vitiligo, eczema, dermatitis or acne are treated.

2. The method of claim 1, wherein selecting said wanted spectrum including at least one of the following spectra: ultraviolet spectrum about (270-320) nm and blue spectrum about (405-435) nm.

3. The method of claim 1, wherein selecting said wanted spectrum by means of wavelength selection including the use of at least one filter which is highly transparent to said wanted spectrum and highly absorbing other spectra.

4. The method of claim 1, wherein selecting said wanted spectrum by means of a wavelength selection including the use of at least one coated mirror having a high reflection in said wanted spectrum and highly transparent to the other spectra at an incident angle of 45 degrees.

5. The method of claim 1 further including wherein the energy per pulse of about (0.2-20) mJ, peak power of about (0.1-5) KW and number of pulses of about (50-1000) in each exposure on the treated area are selected for optimal phototherapy effects.

6. The method of claim 1 wherein said pulse of electromagnetic energy is directed to said skin disorder area by means of a beam delivery.

7. The method of claim 6 said means of beam delivery is an optical fiber being highly transparent to said wanted spectrum.

8. The method of claim 6 said means of beam delivery is a liquid light guide filled with material having highly transparent to said wanted spectrum.

9. The method of claim 6 said means of beam delivery is further connected to a hand piece consisting of at least a spot size controller having an aperture of about (0.5-10) cm to selectively treat both small and large area of said skin disorders with minimum exploring of said wanted light to the adjacent health tissue.

10. An apparatus for the treatment of skin disorders including psoriasis, vitiligo, eczema, dermatitis or acne consists of a power supply, a pulsed incoherent light source, a means of spectra selector, a means of light deliver and a hand piece.

11. The apparatus of claim 10 said means of spectra selector consists of at least one filter or one reflecting mirror to select the wanted UV spectrum about (270-320) nm or blue spectrum about (405-435) nm.

12. The apparatus of claim 10 said pulsed incoherent light has a pulse duration shorter than about 500 microseconds and a repetition rate of (1-500) Hz.

13. The apparatus of claim 10 said means of light deliver consists of an optical fiber or a light guide which is highly transparent to the said wanted spectrum.

14. The apparatus of claim 10 said hand piece consists of at least one of the components of said spectrum selector and said spot size controller providing a light spot size of about (0.5-10) cm on the treated skin surface.
